(19) United States
(12) Patent Application Publication
(10) Pub. No.: US 2008/0188721 A1
(43) Pub. Date: Aug. 7, 2008

(54) METHOD AND APPARATUS FOR IMPLANTABLY ACQUIRING A WIDEBAND SIGNAL

(75) Inventors: Abhilash Patangay, Little Canada, MN (US); Santhosh Seetharaman, Roseville, MN (US); Keith R. Maile, New Brighton, MN (US)

Correspondence Address: SCHWEGMAN, LUNDBERG & WOESSNER, P.A., P.O. BOX 2938, MINNEAPOLIS, MN, 55402, US

(73) Assignee: CARDIAC PACEMAKERS, INC., ST. PAUL, MN (US)

(21) Appl. No.: 11/672,171

(22) Filed: Feb. 7, 2007

(51) Int. Cl. A61B 5/00

(52) U.S. Cl. 600/301

(57) ABSTRACT

This document discusses, among other things, an apparatus and method for implantably acquiring a wideband signal. The apparatus comprises an implantable device including at least one physiological sensor configured to sense physiological signals having a low frequency component and a high frequency component. The implantable device includes a sampling circuit configured to sample at a sampling rate that is lower in frequency than twice the highest frequency of the second frequency component of the physiological signal. The implantable or external signal postprocessing module can be communicatively coupled to the sampling circuit to receive the set of samples, and configured to process the set of samples to use an information of interest intentionally aliased from the second frequency component of the physiological signal.

### TECHNICAL FIELD

This patent document pertains generally to implantable medical devices, and more particularly, but not by way of limitation, to a method and an apparatus for acquiring one or more wideband signals in one or more implanted devices.

### BACKGROUND

Physiological conditions of a subject can provide useful information about the subject's health status, such as to a physician or other caregiver. Implantable medical devices (IMDs) may be implanted within a patient's body for monitoring certain physiological conditions. Some examples of these devices include cardiac function management (CFM) devices such as implantable pacemakers, implantable cardioverter defibrillators (ICDs), cardiac resynchronization devices, and devices that include a combination of such capabilities. CFM devices are typically used to treat patients using electrical or other therapy and to aid a physician or caregiver in patient diagnosis through internal monitoring of a patient's condition. The devices may include one or more electrodes in communication with one or more sense amplifiers to monitor electrical heart activity within a patient, and often include one or more sensors to monitor one or more other internal patient parameters. Other examples of implantable medical devices include implantable diagnostic devices, implantable drug delivery systems, or implantable devices with neural stimulation capability.

### OVERVIEW

The health state of a subject can be evaluated or predicted by using at least one implantable device. Monitoring physiological conditions within a patient's body may benefit from efficiently acquiring or analyzing one or more wideband signals received from one or more physiological sensors. In certain examples, the health state of a subject can be determined by sensing or receiving a wideband signal that includes information about at least one physiological process. Certain signal processing may be performed by the at least one implantable device, or by an external device in communication with the implantable device. Systems and methods for acquiring wideband signals in implantable devices are discussed below.

In Example 1, an apparatus comprises implantable device including a physiological sensor adapted to sense a physiological signal having a first frequency component and a second frequency component, the second frequency component carrying information of interest, and the second frequency component being at a higher frequency than the first frequency component, a continuous-time signal preprocessing circuit, coupled to the physiological sensor to receive the physiological signal, the signal preprocessing circuit configured to output a continuous-time preprocessed physiological signal that represents the second frequency component of the physiological signal, and a sampling circuit, coupled to the signal preprocessing circuit, configured to sample the continuous-time preprocessed physiological signal to generate a set of samples, the sampling circuit configured to sample at a sampling rate that is lower in frequency than twice the highest frequency of the second frequency component of the physiological signal; and an implantable or external signal postprocessing module, communicatively coupled to the sampling circuit to receive the set of samples, and configured to process the set of samples to use the information of interest intentionally aliased from the second frequency component of the physiological signal.

In Example 2, the apparatus of Example 1, is optionally configured such that the sampling rate has a frequency higher than twice the bandwidth of the second frequency component of the physiological signal.

In Example 3, the apparatus of one or both of Examples 1-2, is optionally configured such that the physiological sensor includes at least one of a heart sound sensor, a blood pressure sensor, a cardiac wall motion sensor, a respiration sensor, lung sound sensor and a neural activity sensor.

In Example 4, the apparatus of one or more of Examples 1-3, is optionally configured such that the continuous-time signal preprocessing circuit includes at least one continuous-time filter configured to filter the physiological signal to output the continuous-time preprocessed physiological signal that represents the second frequency component of the physiological signal.

In Example 5, the apparatus of one or more of Examples 1-4, is optionally configured such that the at least one continuous-time filter includes a tunable continuous-time filter.

In Example 6, the apparatus of one or more of Examples 1-5, is optionally configured such that the continuous-time signal preprocessing circuit comprises at least one continuous-time band-pass filtering circuit, and further comprising a time division multiplexing circuit coupled to the sampling circuit.

In Example 7, the apparatus of one or more of Examples 1-6, is optionally configured such that the continuous-time signal preprocessing circuit comprises a mixer circuit configured to combine the physiological signal with an oscillating signal having a center frequency and to shift to a lower frequency the second frequency component, to be output as the continuous-time preprocessed physiological signal that represents the second frequency component of the physiological signal.

In Example 8, the apparatus of one or more of Examples 1-7, comprises a local oscillator configured to generate the oscillating signal.

In Example 9, the apparatus of one or more of Examples 1-8, comprises a physiological event detector adapted to detect a physiological event; and a triggering circuit, coupled to the physiological event detector, the triggering circuit configured to trigger acquisition of the second frequency component of the physiological signal in response to detection of the physiological event.

In Example 10, the apparatus of one or more of Examples 1-9, is optionally configured such that the triggering circuit is configured to trigger acquisition of the second frequency component using information from the first frequency component of the physiological signal.

In Example 11, a method comprises implantably sensing a physiological signal having a first frequency component and a second frequency component, the second frequency component being at a higher frequency than the first frequency component, the second frequency component carrying information of interest; implantably preprocessing the physiological signal in continuous-time for extracting a continuous-time preprocessed physiological signal including the information of interest of the second frequency component; implantably sampling the preprocessed physiological signal to generate a set of samples, the sampling using a sampling frequency that is lower in frequency than twice the highest frequency of the second frequency component of the physiological signal, thereby intentionally aliasing to a lower frequency the information of interest from the second frequency component of the physiological signal; and implantably or externally postprocessing the set of samples to use the intentionally aliased information from the second frequency component of the physiological signal.

In Example 12, the method of Example 11 is optionally configured such that sampling the preprocessed physiological signal to generate a set of samples includes sampling at a sampling rate that has a frequency higher than twice the bandwidth of the second frequency component of the physiological signal.

In Example 13, the method of one or both of Examples 11-12 comprises storing the set of samples in a memory and uploading the set of samples to a programmer device.

In Example 14, the method of one or more of Examples 11-13, is optionally configured such that sensing a physiological signal includes sensing at least one of a thoracic impedance, an intra-cardiac impedance, a heart sound, a blood pressure, a cardiac wall motion, a lung sound, and a neural activity signal.

In Example 15, the method of one or more of Examples 11-14, is optionally configured such that sensing the physiological signal includes sensing an acceleration signal.

In Example 16, the method of one or more of Examples 11-15, is optionally configured such that implantably preprocessing the physiological signal in continuous-time includes filtering the physiological signal to pass the second frequency component of the physiological signal and to attenuate the first frequency component of the physiological signal.

In Example 17, the method of one or more of Examples 11-16, is optionally configured such that implantably preprocessing the physiological signal in continuous-time includes mixing the physiological signal with an oscillating signal having a center frequency, thereby shifting the information of interest in the second frequency component to a lower frequency.

In Example 18, the method of one or more of Examples 11-17, comprises detecting a physiological event using a physiological event detector; and triggering acquisition of the second frequency component of the physiological signal in response to detection of the physiological event.

In Example 19, the method of one or more of Examples 11-18, comprises triggering acquisition of the second frequency component using information acquired from the first frequency component of the physiological signal.

In Example 20, an apparatus comprises means for implantably sensing a physiological signal having a first frequency component and a second frequency component, the second frequency component being at a higher frequency than the first frequency component, the second frequency component carrying information of interest; means for implantably preprocessing the physiological signal in continuous-time for extracting a continuous-time preprocessed physiological signal including the information of interest of second frequency component; means for implantably sampling the preprocessed physiological signal to generate a set of samples, the sampling using a sampling frequency that is lower in frequency than twice the highest frequency of the second frequency component of the physiological signal, thereby intentionally aliasing to a lower frequency the information of interest from the second frequency component of the physiological signal; and means for implantably or externally postprocessing the set of samples to use the intentionally aliased information from the second frequency component of the physiological signal.

In Example 21, the apparatus of Example 20, is optionally configured to sample at a sampling frequency that is higher than twice the bandwidth of the second frequency component of the physiological signal.

In Example 22, the apparatus of one or both of Example 20-21, is optionally configured such that the means for implantably sensing a physiological signal includes at least one of a heart sound sensor, a blood pressure sensor, a cardiac wall motion sensor, a respiration sensor, a lung sound sensor and a neural activity sensor configured to sense a neural activity signal.

In Example 23, the apparatus of one or more of Examples 20-22, is optionally configured such that the means for implantably preprocessing the physiological signal includes at least one continuous-time filter configured to pass the second frequency component of the physiological signal and configured to attenuate the first frequency component of the physiological signal.

In Example 24, the apparatus of one or more of Examples 20-23, is optionally configured such that at least one continuous-time filter includes a tunable continuous time-filter.

In Example 25, the apparatus of one or more of Examples 20-24, is optionally configured such that the means for implantably preprocessing the physiological signal comprises a mixer circuit configured to combine the physiological signal with an oscillating signal having a center frequency and to shift the second frequency component to a lower frequency.

In Example 26, the apparatus of one or more of Examples 20-25, comprises a means for triggering an acquisition of the second frequency component of the physiological signal based on detected physiological event.

In Example 27, the apparatus of one or more of Examples 20-26, comprises a means for triggering an acquisition of the second frequency component of the physiological signal using information from the first frequency component of the physiological signal.

This overview is not intended to be an exclusive or exhaustive treatment of the present subject matter. Further details are found in the detailed description and appended claims. Other aspects may be apparent to persons skilled in the art upon reading and understanding the following detailed description and viewing the drawings that form a part thereof, each of which are not to be taken in a limiting sense. The scope of the present invention is defined by the appended claims and their equivalents.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a diagram illustrating one conceptual example of a frequency spectrum of a wideband physiological sensor output signal that represents a physiological parameter in a subject.

FIG. 2 is a diagram illustrating one conceptual example of a frequency spectrum of a filtered wideband physiological sensor output signal shown in FIG. 2.

FIG. 3 is a diagram illustrating one conceptual example of a frequency spectrum shown in FIG. 3 aliased to a lower frequency band.

FIG. 4 is a schematic view illustrating a system adapted to predict monitor, or treat an occurrence of impending heart failure or other disease state in a subject.

FIG. 5 is a block diagram illustrating one conceptual example of an implantable medical device (IMD) having physiological sensors configured to provide wideband physiological sensor output signals that may be used to predict, monitor, or treat an occurrence of impending heart failure or other disease state in a subject.

FIG. 6 is a block diagram illustrating one conceptual example of a signal processing circuit used to extract a high frequency component signal from a wideband physiological sensor output signal that represents a physiological parameter in a subject.

FIG. 7 is a block diagram illustrating another conceptual example of a signal processing circuit used to extract a high frequency component signal from a wideband physiological sensor output signal that represents a physiological parameter in a subject.

FIG. 8 is a block diagram illustrating yet another conceptual example of a signal processing circuit used to extract a high frequency component signal from a wideband physiological sensor output signal that represents a physiological parameter in a subject.

FIG. 9 is a block diagram illustrating yet another conceptual example of a signal processing circuit used to extract a high frequency component signal from a wideband physiological sensor output signal that represents a physiological parameter in a subject.

FIG. 10 is a flow chart illustrating generally, one example of a method of extracting high frequency component signals from a wideband physiological sensor output signal that represents a physiological parameter in a subject.

### DETAILED DESCRIPTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, logical and electrical changes may be made without departing from the scope of the present invention. The following detailed description is, therefore, not to be taken in a limiting sense, and the scope of the present invention is defined by the appended claims and their equivalents.

In this document, the terms “a” or “an” are used, as is common in patent documents, to include one or more than one. In this document, the term “or” is used to refer to a nonexclusive or, such that “A or B” includes “A but not B.” “B but not A,” and “A and B,” unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

An implantable medical device (IMD) may include one or more of the features, structures, methods, or combinations thereof described herein. For example, a cardiac monitor or a cardiac stimulator may be implemented to include one or more of the advantageous features or processes described below. It is intended that such a monitor, stimulator, or other implantable or partially implantable device need not include all of the features or processes described herein.

### EXAMPLES

The present systems and methods may be used in applications involving implantable medical devices (“IMDs”) including, but not limited to, implantable cardiac function management (“CFM”) systems such as pacemakers, cardioverters/defibrillators, pacemakers/defibrillators, biventricular or other multi-site resynchronization or coordination devices such as cardiac resynchronization therapy (“CRT”) devices, patient monitoring systems, neural modulation systems, or drug delivery systems, or devices including one or more combinations of such functionality. In addition, the systems and methods described herein may also be employed in unimplanted devices, including but not limited to, external pacemakers, neutral stimulators, cardioverters/defibrillators, pacer/defibrillators, biventricular or other multi-site resynchronization or coordination devices, monitors, programmers and recorders, whether such devices are used for providing sensing, receiving, prediction processing, or therapy.

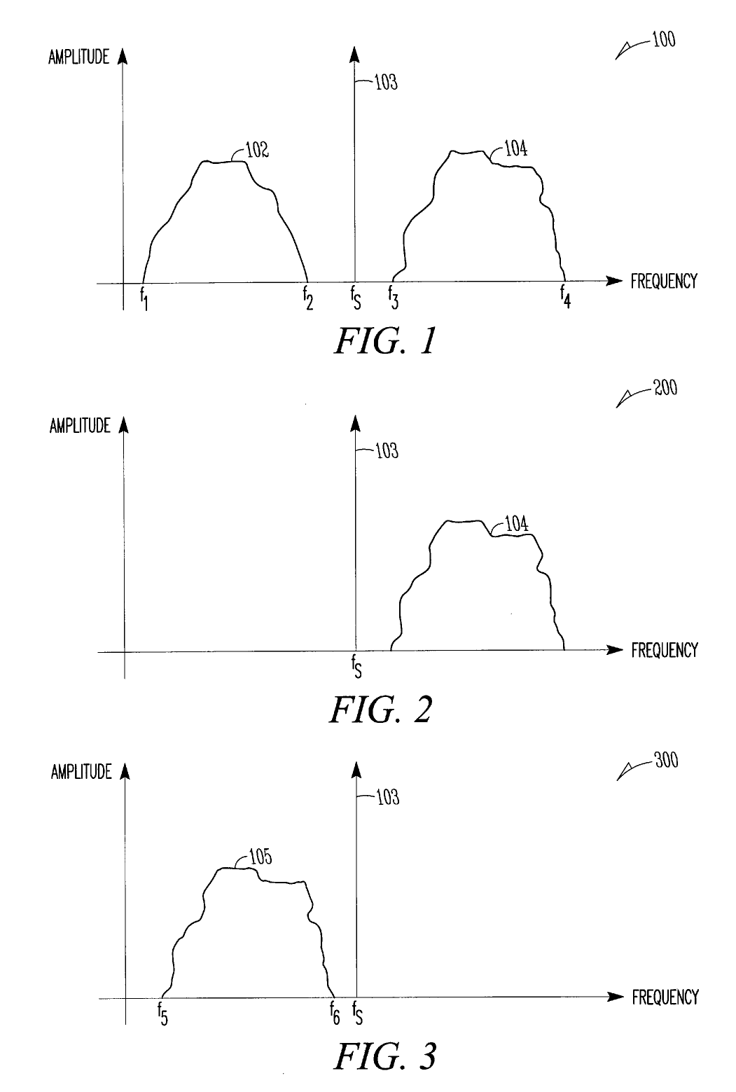

FIG. 1 is a diagram illustrating one conceptual example of a frequency spectrum 100 of a wideband physiological sensor output signal that represents a physiological parameter in a subject. FIG. 1 shows a frequency spectrum of a low frequency component 102, a high frequency component 104 and a sampling frequency fₛ 103 used by a sampling circuit. Low frequency component 102 spans between frequencies f₁ Hz and f₂ Hz. High frequency component 104 spans between frequencies f₃ Hz and f₄ Hz. In one example, low frequency component 102 spans between about 0 Hz and about 90 Hz, and high frequency component 104 spans between about 100 Hz and about 1 kHz. In this example, the sampling frequency fₛ Hz is greater than the highest frequency of low frequency component 102 and is lesser than the lowest frequency of high frequency component 104.

FIG. 2 is a diagram illustrating one conceptual example of a frequency spectrum 200 of a filtered physiological sensor output signal shown in FIG. 3. As shown in FIG. 2, low frequency component 102 is filtered out, such as by a signal processing circuit and high frequency component 104 is passed through.

FIG. 3 is a diagram 300 illustrating one conceptual example of high frequency component 104 shown in FIG. 2 intentionally aliased to a lower frequency band 105. According to the Nyquist-Shannon sampling theorem, a band-limited signal x(t) whose frequency spectrum (ranging between a lower frequency f₁ to an upper frequency f₂) ranges over a frequency bandwidth B Hz (obtained by subtracting f₂ and f₁) can be reconstructed perfectly from its sampled version x[n], if the sampling rate fₛ is more than twice the frequency bandwidth (B Hz) of the band-pass signal x(t). In situations where sampling frequency fₛ falls within the boundaries given by the equation, B<fₛ<2B, a phenomenon of aliasing is exhibited. As the sampling condition laid out by sampling theorem is not satisfied, the frequency components within the frequency spectrum will overlap. The frequency components above half the sampling rate fₛ will be reconstructed at, and will appear as, frequencies below half the sampling rate fₛ. This is called aliasing and the reconstructed signal is said to be an alias of the original signal, in the sense that it corresponds to the same set of frequency sample values (as opposed to time sample values).

Aliasing can be used for down-sampling the high-frequency component that includes a signal of interest. By intentionally aliasing the high frequency component 104 (by using a sampling frequency lower than twice the bandwidth of high frequency component 104), the frequency spectrum of high frequency component 104 is shifted below frequency fₛ.

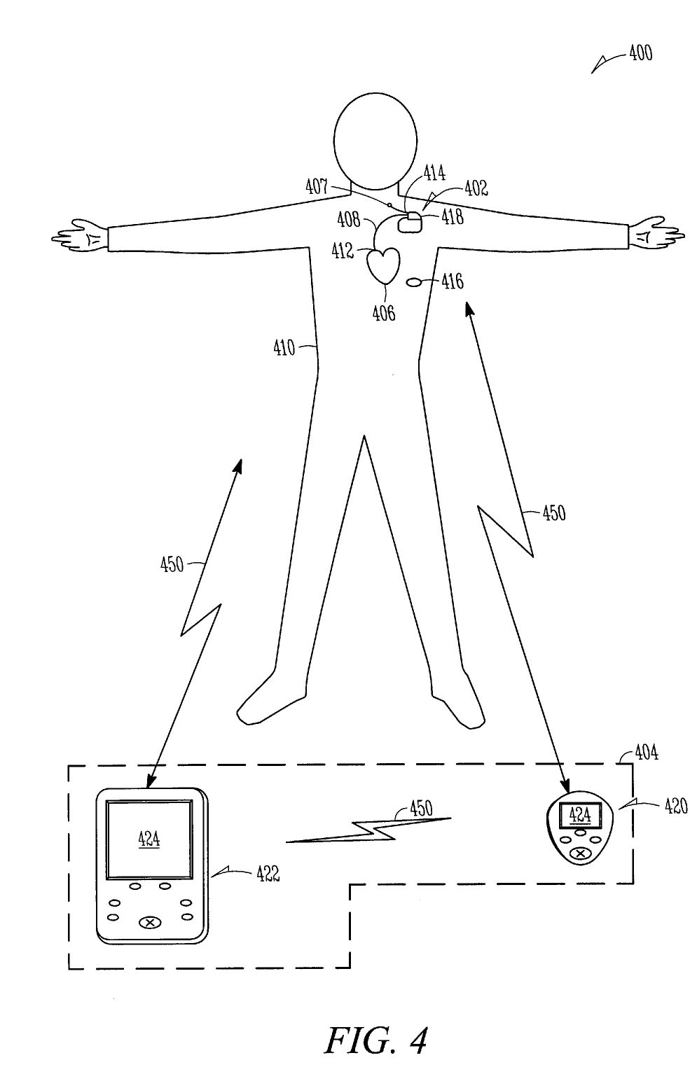

FIG. 4 is a schematic view illustrating an example of a system 400 adapted to predict, monitor, or treat a physiological condition or disease state (e.g., heart failure, etc.) in a subject 410, such as by using one or more wideband physiological signals sensed at one or more physiological sensors. In the example shown in FIG. 4, the system 400 includes an IMD 402, such as a CFM device, which can be coupled by at least one lead 408 to a heart 406 or nerve (such as an efferent parasympathetic nerve, e.g., a vagus nerve 407), of the subject 410. The IMD 402 may be implanted subcutaneously, such as in the subject's chest, abdomen, or elsewhere. In this example, lead 408 extends from a lead proximal portion 414 to a lead distal portion 412.

In the example of FIG. 4, system 400 can also optionally include one or more of an external device 404, one or more remote portions (e.g., a nearby or local external user-interface 420 or a distant or remote external user interface 422, which may use a local repeater and a communications network), a drug dispenser 416, or a warning device 418. The remote portions 420, 422 of the external device 404 may provide direct or indirect wireless communication with the IMD 402, such as by using telemetry 450, and may provide direct or indirect wired or wireless communication with each other. In certain examples, the prediction, monitoring, or treatment of a physiological condition or disease state can be made, at least in part, by receiving, communicating, or processing information about at least one physiological sensor producing a high frequency signal. In certain examples, one or more remote portions of the external device 404 include a visual or other display 424, such as for textually or graphically relaying information to the subject 410 or a caregiver.

The drug dispenser 416 may optionally be provided to automatically deliver or evaluate the efficacy of a diuretic, drug, or other substance, such as based on collected physiological information. The physiological information can also be used to control or evaluate efficacy of one or more other therapies, such as neurostimulation therapy, for example, or to provide a warning about an impending physiological condition occurring, such as during a specified future prediction time period. One or more warning signals may be generated using either an internal warning device 418 or the external user interfaces 420, 422.

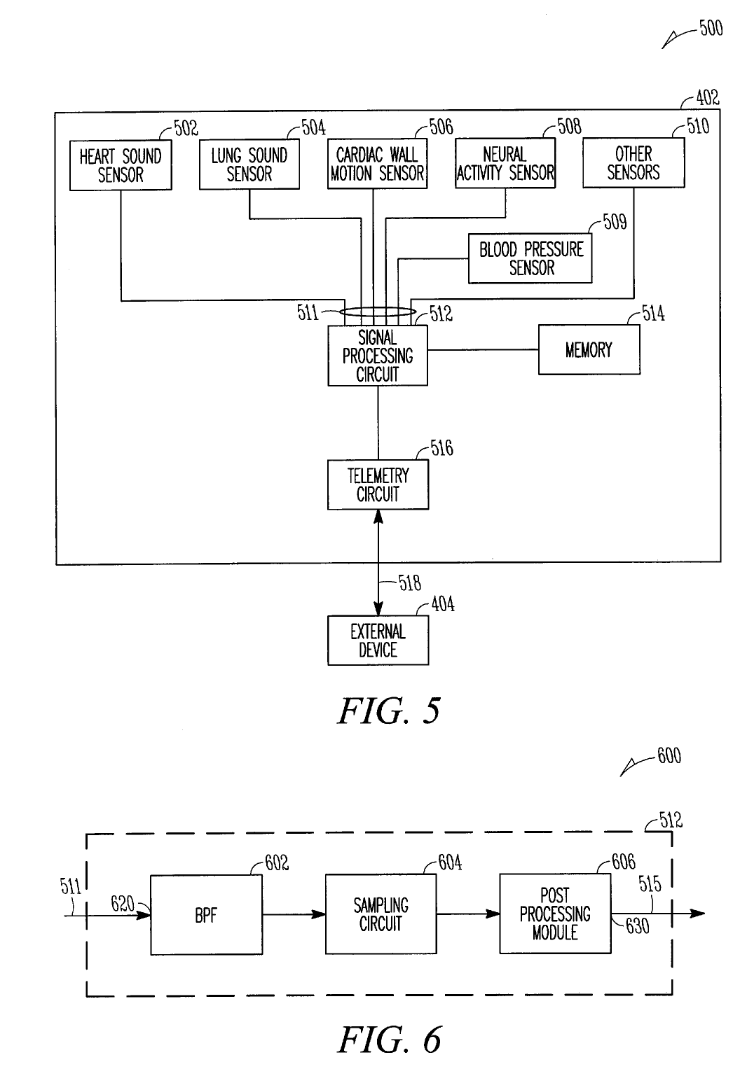

FIG. 5 is a block diagram 500 illustrating an example of IMD 402 (FIG. 4) having one or more physiological sensors configured to sense one or more respective wideband physiological signals. In this example, IMD 402 includes one or more physiological sensors 502-510, a signal processing circuit 512, a memory 514, and a telemetry circuit 516. In one example, the physiological sensors available in IMD 402 can include one or more of a heart sound sensor 502, a lung sound sensor 504, a cardiac wall motion sensor 506, a neural activity sensor 508, a blood pressure sensor 509 or one or more other sensors 510. In the illustrative example shown, each of the physiological sensors 502-510 is coupled to signal processing circuit 512. Signal processing circuit 512 is coupled to a memory 514 and a telemetry circuit 516. In certain examples, IMD 402 is typically communicatively coupled to a external device 404 (FIG. 1) using wireless telemetry circuit 516.

Signal processing circuit 512 is configured to receive one or more of the various wideband signals received at one or more physiological sensors 502-510, such as to determine a change in at least one physiological parameter of the subject 410. In certain examples, signal processing circuit 512 stores received data from one or more physiological sensors 502-510, such as in memory 514. In certain examples, signal processing circuit 512 stores processed physiological parameter information in memory 514. Physiological parameter information can also be communicated by telemetry circuit 516 over a telemetry link 518, such as to external device 404.

FIG. 6 is a block diagram 600 illustrating a conceptual example of a signal processing circuit 512 that can be used to extract a high frequency component signal of a physiological sensor output signal that represents a physiological parameter in a subject 410. In this example, circuit 512 includes a band-pass filter 602, a sampling circuit 604 and a postprocessing module 606. Band-pass filter 602 includes input port 620 and postprocessing 606 includes output port 630.

Input port 620 is coupled to at least one of the physiological sensors 502-510 (FIG. 5), such as by using connection link 511. Typically, the input signal on connection link 511 is a wideband signal that includes both a low frequency component and a high frequency component. The high frequency component corresponds to physiological information of interest sensed by one or more physiological sensors 502-510. In certain examples, band-pass filter 602 is a continuous-time filter configured to pass or even amplify a particular portion of the frequency spectrum of the input signal on connection link 511 received at input port 620. Many different implementations of band-pass filter 602 may be suitable. In certain examples, bandpass filter 602 includes a switched-capacitor bi-quadratic filter stage, series-coupled with a subsequent switched-capacitor gain stage. In certain examples, the filter bandwidth or gain of the band-pass filter 602 is tunable, such as by setting the capacitance values.

Band-pass filter 602 is coupled to the sampling circuit 604. Sampling circuit 604 is configured to receive a continuous-time signal x(t) from band-pass filter 602 and to convert the same into a discrete-time digital signal x[n] to be provided to postprocessing module 606. Typically, sampling circuit 604 samples the continuous-time signal x(t) and generates a discrete-time digital signal x[n], such as by measuring the value of the continuous-time signal x(t) every T seconds. As a result, sampled signal x[n] is given by x[n]=x(nT), where, n=0,1,2,3, . . . . Additionally, a sampling frequency or sampling rate fₛ represents the number of samples obtained in one second, or fₛ=1/T.

Sampling circuit 604 is coupled to the postprocessing module 606. Output port 630 is coupled to telemetry circuit 516 (FIG. 5), such as by using connection link 515. Postprocessing module 606 is configured to reconstruct the sampled discrete-time signal x[n] to form a signal representing x(t). In certain examples, postprocessing module 606 is configured to detect a change in one or more signal characteristics of a signal sensed by the one or more physiological sensors 504-510. The postprocessing module 606 may also be configured to compensate for the intentional aliasing performed at sampling circuit 604.

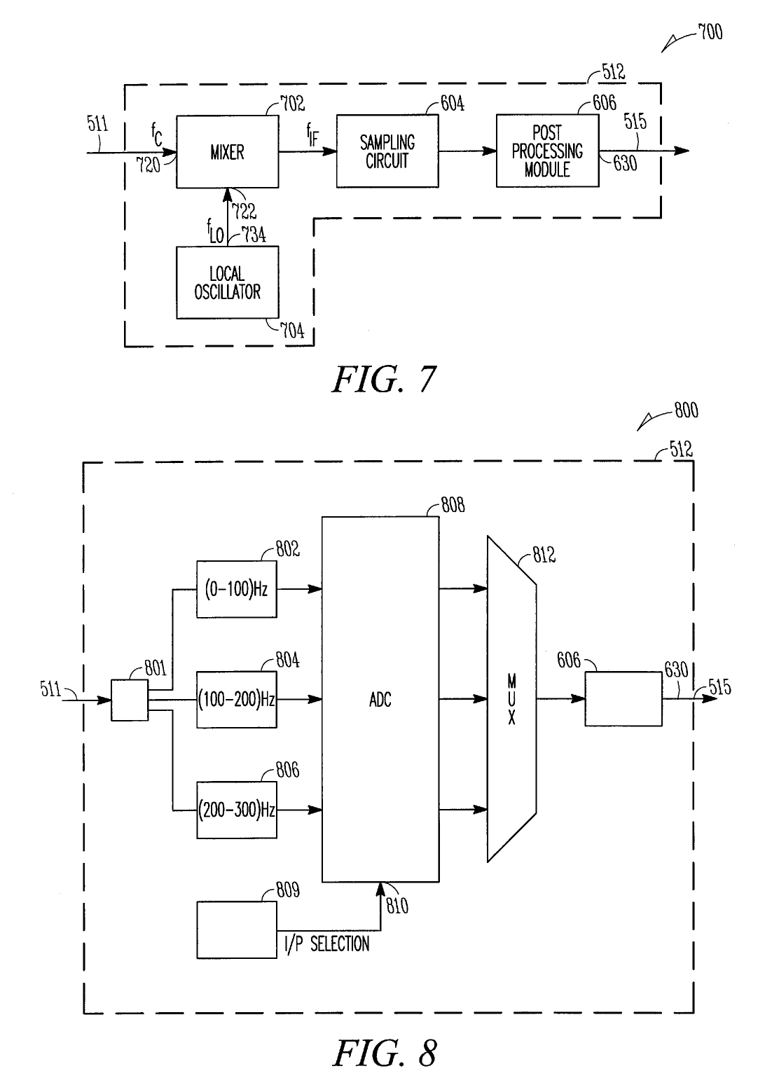

FIG. 7 is a block diagram 700 illustrating another conceptual example of a signal processing circuit 512 used to extract a high frequency component of a physiological sensor output signal. In this example, the signal processing circuit 512 includes a mixer 702, a local oscillator 704, a sampling circuit 604 (FIG. 6) and a postprocessing module 606 (FIG. 6). Mixer 702 includes input ports 720 and 722. Local oscillator 704 includes output port 734. As described before, postprocessing module 606 includes an output port 630, which is coupled to telemetry circuit 516 (FIG. 5), such as by using connection link 515.

Input port 720 is coupled to at least one of the physiological sensors 502-510 (FIG. 5), such as by using connection link 511. The input signal on connection link 511 includes a high frequency component that corresponds to one or more physiological parameters represented in the signal.

Output port 734 of local oscillator 704 is coupled to the input port 722 of mixer 702. Local oscillator 704 generates an oscillating signal having an oscillating frequency f_{LO}. Typically, the frequency of the signal at output of mixer 702 is given by relation f_{IF}=|f_{LO}−f_{c}|, where f_{c} is the frequency of the incoming signal at input port 720 of mixer 702 and f_{IF} is the frequency of the signal at output of mixer 702. In one example, the center frequency of the oscillating signal generated by local oscillator 704 is adjustable, thereby enabling retrieval of a desired frequency band from the input signal on connection link 511.

The mixer 702 is coupled to the sampling circuit 604. The sampling circuit 604 is coupled to postprocessing module 606, such as described earlier. In certain examples, sampling circuit 604 is configured to receive a continuous-time signal x(t) with frequency f_{IF} from the output of mixer 702, and is configured to convert the same into a discrete-time digital signal x[n] to be provided an output to postprocessing module 606 as described above under FIG. 6. The sampling circuit 604 can also provide decimation (reduction in number of samples).

FIG. 8 is a block diagram 800 illustrating yet another conceptual example of a signal processing circuit 512 that can be used to extract a high frequency component signal of a physiological sensor output signal. In certain examples, signal processing circuit 512 includes a splitter 801, two or more band-pass filters 802, 804, and 806, an analog-to-digital converter (ADC) 808, a multiplexer 812, and a postprocessing module 606 (FIG. 6, FIG. 7). In certain examples, one or more additional band-pass filters may be used to extract particular frequencies bands from the incoming signal. ADC 808 also typically includes sampling and decimation hardware, such as may be available in sampling circuit 604.

The splitter 801 is coupled to at least one of the physiological sensors 502-510 (FIG. 5), such as by using connection link 511. The band-pass filters 802, 804, and 806 are coupled to splitter 801. The ADC 808 is coupled to the band-pass filters 802, 804, and 806. The multiplexer 812 is coupled to the ADC 808. Postprocessing module 606 is coupled to the multiplexer 812. In certain examples, multiplexer 812 is a time-division multiplexer that is configured to collect digital samples from the ADC 808 to be processed at postprocessing module 606. As described before, postprocessing module 606 includes an output port 630, which is coupled to telemetry circuit 516 (FIG. 5), such as by using connection link 515.

The band-pass filters 802, 804, and 806 are configured to operate over certain desired frequency bands. In certain examples, an input selection signal is received at a communication port 810 of ADC 808 which allows for the selection of desired frequency bands to be monitored. In certain examples, the input selection signal is generated at a triggering circuit 809. In certain examples, the triggering circuit 809 sends the input selection signal based on monitoring one or more low frequency component signals. Information in a low frequency component signal can therefore act as a triggering event, such as for selection of a particular bandpass filter. One example of a triggering event is the occurence of a myocardial infarction (MI), which may be determined by monitoring a low frequency component of the wideband signal on connection link 511. Such a triggering event can initiate the intentional aliasing and capture of one or more high frequency component signals from the incoming wideband signal on connection link 511. Other examples of triggering physiological events that can be captured using low frequency components within the incoming wideband signal at connection link 511 may include increasing blood pressure, lung pressure etc.

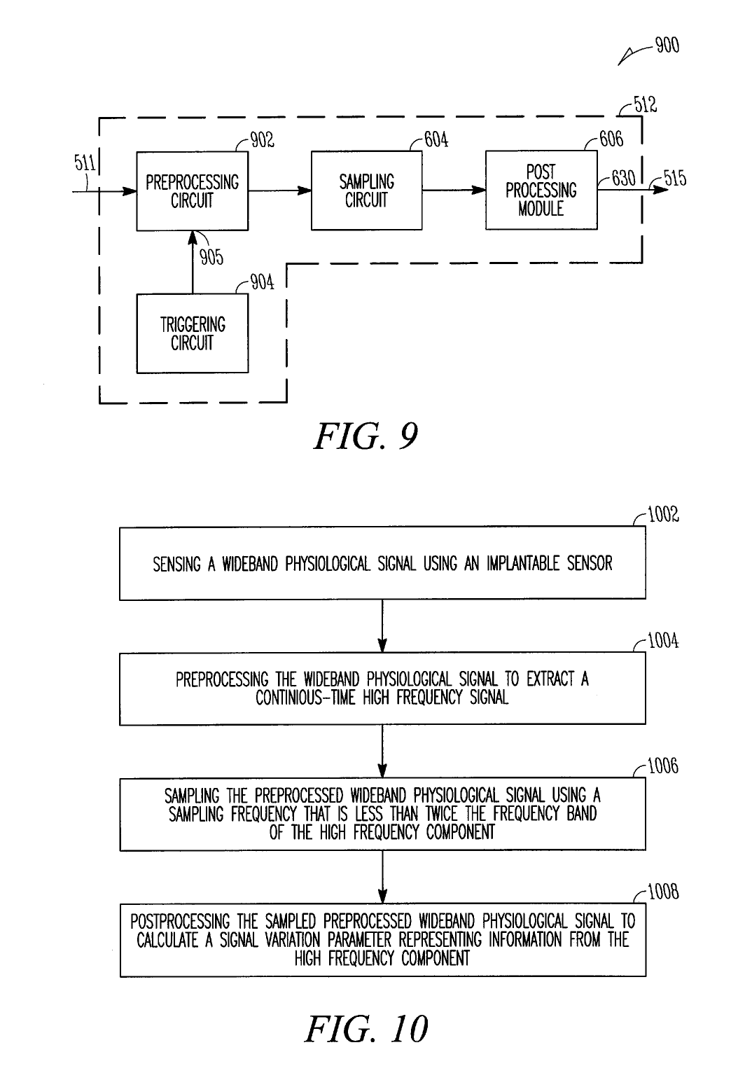

FIG. 9 is a block diagram 900 illustrating another conceptual example of a signal processing circuit 512 that can be used to extract a high frequency component signal of a physiological sensor output signal. In certain examples, signal processing circuit 512 includes a preprocessing circuit 902, a triggering circuit 904, a sampling circuit 604, and a postprocessing module 606 (FIG. 6, FIG. 7, FIG. 8). In certain examples, the preprocessing circuit 902 includes at least one bandpass filter configured to operate over certain desired frequency bands. In certain examples, the preprocessing circuit 902 includes a mixer and a local oscillator.

The preprocessing circuit 902 is coupled to at least one of the physiological sensors 502-510 (FIG. 5), such as by using connection link 511. The triggering circuit 904 is coupled to the preprocessing circuit 902 at communication port 905. The sampling circuit 604 is coupled to the preprocessing circuit 902. The postprocessing module 606 is coupled to the sampling circuit 604. As described before, the postprocessing module 606 includes an output port 630, which is coupled to telemetry circuit 516 (FIG. 5), such as by using connection link 515.

In certain examples, a triggering signal is generated at the triggering circuit 904 and received at the communication port 905 of preprocessing circuit 902. In certain examples, the triggering circuit 904 sends the triggering signal based on monitoring one or more low frequency component signals. Information in a low frequency component signal can therefore act as a triggering event, such as for monitoring high frequency component signals.

FIG. 10 is a flow chart illustrating generally, one example of a method of extracting a continuous-time signal representing a high frequency component of a physiological signal in subject 410.

At 1002, at least one physiological signal is sensed or received using at least one implantable sensor, such as one of the implantable sensors 502-510. In certain examples, the sensor provides a wideband physiological signal having a low frequency component 102 and a high frequency component 104, which is received at input 620 of band-pass filter 602 (FIG. 6). In certain examples, the sensor provides the wideband physiological signal having low frequency component 102 and high frequency component 104, which is received at input 720 of mixer 702 (FIG. 7). In certain examples, the sensor provides the wideband physiological signal having low frequency component 102 and high frequency component 104, which is received at splitter 801 (FIG. 8). Examples of physiological signals sensed and received at 1002 can include one or more of: thoracic impedance, intra-cardiac impedance, heart sounds, cardiac wall motion, lung sounds, neural activity signals, acceleration, etc.

At 1004, the one or more physiological signals are preprocessed to extract a continuous-time high frequency signal x(t). In certain examples, band-pass filter 602 attenuates the low frequency component 102 and passes high frequency component 104 of the physiological signal (FIG. 6). In certain examples, mixer 702 and local oscillator 734 are configured to retrieve a desired frequency band from the input signal on connection link 511 (FIG. 7).

At 1006, the one or more preprocessed continuous time physiological signals is sampled at sampling circuit 604. The sampling is performed using a sampling frequency that is lower than the high frequency component 104. This intentionally aliases the incoming high frequency component 104 to shift its information to a lower frequency.

At 1008, the intentionally aliased (down-shifted) signal is received at a postprocessing module 606, which processes its input signal to use the intentionally aliased information of interest from the second frequency component 104 of the wideband physiological signal. In certain examples, compensation for the intentional aliasing performed at sampling circuit 604 is provided at postprocessing module 606.

The following discussion provides examples of physiological sensors 502-508 providing wideband signals having high frequency components whose presence, absence, or baseline change is statistically associated with an occurrence of impending heart failure; the list is not meant to be exhaustive, and may include other sensors 510 not discussed herein.

Heart Sound Sensor

A heart sound sensor 502 can be configured to sense information indicative of one or more heart sounds of subject 410. A “heart sound” can include a first heart sound (“S1”), a second heart sound (“S2”), a third heart sound (“S3”), a fourth heart sound (“S4”), or any components thereof, such as the aortic component of S2 (“A2”), the pulmonary component of S2 (“P2”), or other broadband sounds or vibrations associated with valve closures or fluid movement, such as a heart murmur, etc. Heart sounds can also include one or more broadband chest sounds, such as may result from one or more of mitral regurgitation, left ventricle dilation, etc. The heart sound sensor 502 typically provides an electrical “heart sound signal” that includes heart sound information. The heart sound sensor 502 can include any device configured to sense the heart sound signal of the subject. In certain examples, the heart sound sensor 502 can include an implantable sensor including at least one of an accelerometer, an acoustic sensor, a microphone, etc. In certain examples, heart sound sensor 502 is used to sense a heart sound signal that includes a low frequency and a high frequency component. When the heart sound changes from a baseline, it may be associated with impending heart failure. Generally, heart sounds of interest include murmurs and other indications of valvular disease. Typically, the frequency range of desired heart sounds include from about 2 Hz to about 1000 Hz.

In certain examples, where the heart sound signal may be from mitral regurgitation, the physiological signal can have a frequency band in the range of about 250 Hz to about 800 Hz. In certain examples, the frequency band of interest may only be between about 500 Hz and 700 Hz. The sampling frequency is chosen such that it is more than twice the bandwidth of the frequency band of interest.

Additionally, the sampling frequency is chosen to be less than twice the highest frequency component of the frequency band of interest. Therefore, in the example above a sampling frequency of 400 Hz used in signal processing circuit 512 will enable the extraction of desired signal energies within the frequency band between 500 Hz and 700 Hz. One of the many advantages of using signal processing circuit 512 include the ability of using sampling frequencies that are lower than twice the highest frequency of the frequency band of desired physiological signals to extract desired physiological signals. Furthermore, the use of a lower sampling frequency in signal processing circuit 512 allows for a smaller power budget for implantable devices such as IMD 402.

Lung Sound Sensor

In another example, lung sounds sensor 504 can be configured to sense a signal representing the lung sound of subject 410. Broad-band chest sounds are useful for heart failure patient management. Detection of lung sounds helps the implantable device to provide more appropriate and optimal therapy. The lung sound signal can include any signal indicative of at least a portion of at least one lung sound of the subject. The subject's changed pulmonary (lung) sounds (e.g., increased rales and wheezes) is used as a physiologic parameter that is statistically associated with impending thoracic fluid accumulation and/or lung congestion. In one example, an increase in the frequency or amplitude of rates may correlate to a future thoracic fluid accumulation. Additionally, recording these sounds and uploading the over an advanced patient management infrastructure can help a doctor to give better disease management.

Cardiac Wall Motion Sensor

In another example, cardiac wall motion sensor 506 can be configured to sense during each cardiac cycle, a signal representing the inward and outward displacement of the ventricular endocardial wall of subject 410.

Neural Activity Sensor

In another example, neural activity sensor 508 can be configured to sense a signal representing the nerve activity in or around the pulmonary artery by at least one electrode intravascularly inserted into the pulmonary artery of subject 410.

Blood Pressure Sensor

In another example, a blood pressure sensor 509 can be configured to sense a signal representing the pressure inside at least one of the atriums or ventricles of the heart, pulmonary artery or any blood vessel of subject 410.

Heart failure is a common entity, particularly among the elderly, but it often not treated (if at all) until the disease is detected late in the disease process via associated symptoms, such as abnormal thoracic fluid build-up behind the heart. Advantageously, the present systems and methods allow for the prediction, monitoring, or treatment of impending heart failure or other disease states by monitoring one or more high frequency components of signals associated with a subject's physiological process. Sensing wideband physiological signals in the human body provides a means for monitoring, predicting or treating an impending disease state, such as heart failure or lung failure among others.

It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to comply with 37 C.F.R. §1.72(b), which requires that it allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment.

What is claimed is:

1. An apparatus, comprising:
an implantable device, including:
a physiological sensor adapted to sense a physiological signal having a first frequency component and a second frequency component, the second frequency component carrying information of interest, and the second frequency component being at a higher frequency than the first frequency component;
a continuous-time signal preprocessing circuit, coupled to the physiological sensor to receive the physiological signal, the signal preprocessing circuit configured to output a continuous-time preprocessed physiological signal that represents the second frequency component of the physiological signal; and
a sampling circuit, coupled to the signal preprocessing circuit, configured to sample the continuous-time preprocessed physiological signal to generate a set of samples, the sampling circuit configured to sample at a sampling rate that is lower in frequency than twice the highest frequency of the second frequency component of the physiological signal; and
an implantable or external signal postprocessing module, communicatively coupled to the sampling circuit to receive the set of samples, and configured to process the set of samples to use the information of interest intentionally aliased from the second frequency component of the physiological signal.

2. The apparatus of claim 1, wherein the sampling circuit is configured to sample at a sampling rate that has a frequency higher than twice the bandwidth of the second frequency component of the physiological signal.

3. The apparatus of claim 2, wherein the physiological sensor includes at least one of a heart sound sensor, a blood pressure sensor, a cardiac wall motion sensor, a respiration sensor, lung sound sensor and a neural activity sensor.

4. The apparatus of claim 2, wherein the continuous-time signal preprocessing circuit includes at least one continuous-time filter configured to filter the physiological signal to output the continuous-time preprocessed physiological signal that represents the second frequency component of the physiological signal.

5. The apparatus of claim 4, wherein the at least one continuous-time filter includes a tunable continuous-time filter.

6. The apparatus of claim 2, wherein the continuous-time signal preprocessing circuit comprises at least one continuous-time band-pass filtering circuit, and further comprising a time division multiplexing circuit coupled to the sampling circuit.

7. The apparatus of claim 2, wherein the continuous-time signal preprocessing circuit comprises:
a mixer circuit configured to combine the physiological signal with an oscillating signal having a center frequency and to shift to a lower frequency the second frequency component, to be output as the continuous-time preprocessed physiological signal that represents the second frequency component of the physiological signal.

8. The apparatus of claim 7, comprising a local oscillator configured to generate the oscillating signal.

9. The apparatus of claim 1, comprising:
a physiological event detector adapted to detect a physiological event; and
a triggering circuit, coupled to the physiological event detector, the triggering circuit configured to trigger acquisition of the second frequency component of the physiological signal in response to detection of the physiological event.

10. The apparatus of claim 9, wherein the triggering circuit is configured to trigger acquisition of the second frequency component using information from the first frequency component of the physiological signal.

11. A method comprising:
implantably sensing a physiological signal having a first frequency component and a second frequency component, the second frequency component being at a higher frequency than the first frequency component, the second frequency component carrying information of interest;
implantably preprocessing the physiological signal in continuous-time for extracting a continuous-time preprocessed physiological signal including the information of interest of the second frequency component;
implantably sampling the preprocessed physiological signal to generate a set of samples, the sampling using a sampling frequency that is lower in frequency than twice the highest frequency of the second frequency component of the physiological signal, thereby intentionally aliasing to a lower frequency the information of interest from the second frequency component of the physiological signal; and
implantably or externally postprocessing the set of samples to use the intentionally aliased information from the second frequency component of the physiological signal.

12. The method of claim 11, wherein implantably sampling the preprocessed physiological signal to generate a set of samples includes sampling at a sampling rate that has a frequency higher than twice the bandwidth of the second frequency component of the physiological signal.

13. The method of claim 12, further comprising storing the set of samples in a memory and uploading the set of samples to a programmer device.

14. The method of claim 12, wherein sensing a physiological signal includes sensing at least one of a thoracic impedance, an intra-cardiac impedance, a heart sound, a blood pressure, a cardiac wall motion, a lung sound, and a neural activity signal.

15. The method of claim 12, wherein sensing the physiological signal includes sensing an acceleration signal.

16. The method of claim 12, wherein implantably preprocessing the physiological signal in continuous-time includes filtering the physiological signal to pass the second frequency component of the physiological signal and to attenuate the first frequency component of the physiological signal.

17. The method of claim 12, wherein implantably preprocessing the physiological signal in continuous-time includes mixing the physiological signal with an oscillating signal having a center frequency, thereby shifting the information of interest in the second frequency component to a lower frequency.

18. The method of claim 12, further comprising:
detecting a physiological event using a physiological event detector; and
triggering acquisition of the second frequency component of the physiological signal in response to detection of the physiological event.

19. The method of claim 12, further comprising:
triggering acquisition of the second frequency component using information acquired from the first frequency component of the physiological signal.

20. An apparatus comprising:
means for implantably sensing a physiological signal having a first frequency component and a second frequency component, the second frequency component being at a higher frequency than the first frequency component, the second frequency component carrying information of interest;
means for implantably preprocessing the physiological signal in continuous-time for extracting a continuous-time preprocessed physiological signal including the information of interest of second frequency component;
means for implantably sampling the preprocessed physiological signal to generate a set of samples, the sampling using a sampling frequency that is lower in frequency than twice the highest frequency of the second frequency component of the physiological signal, thereby intentionally aliasing to a lower frequency the information of interest from the second frequency component of the physiological signal; and
means for implantably or externally postprocessing the set of samples to use the intentionally aliased information from the second frequency component of the physiological signal.

21. The apparatus of claim 20, wherein the sampling frequency is higher than twice the bandwidth of the second frequency component of the physiological signal.

22. The apparatus of claim 21, wherein the means for implantably sensing a physiological signal includes at least one of a heart sound sensor, a blood pressure sensor, a cardiac wall motion sensor, a respiration sensor, a lung sound sensor and a neural activity sensor configured to sense a neural activity signal.

23. The apparatus of claim 21, wherein the means for implantably preprocessing the physiological signal includes at least one continuous-time filter configured to pass the second frequency component of the physiological signal and configured to attenuate the first frequency component of the physiological signal.

24. The apparatus of claim 23, wherein at least one continuous-time filter includes a tunable continuous time-filter.

25. The apparatus of claim 21, wherein the means for implantably preprocessing the physiological signal comprises:
a mixer circuit configured to combine the physiological signal with an oscillating signal having a center frequency and to shift the second frequency component to a lower frequency.

26. The apparatus of claim 21, comprising:
means for triggering an acquisition of the second frequency component of the physiological signal based on detected physiological event.

27. The apparatus of claim 21, comprising:
means for triggering an acquisition of the second frequency component of the physiological signal using information from the first frequency component of the physiological signal.
